(19) United States
(12) Patent Application Publication
(10) Pub. No.: US 2006/0190079 A1
(43) Pub. Date: Aug. 24, 2006

(54) Articulating spinal disc implants with amorphous metal elements

(76) Inventors: Naim Istephanous, Roseville, MN (US); Joe Lessar, Coon Rapids, MN (US); Darrel Untereker, Oak Grove, MN (US); Lukas Eisermann, Memphis, TN (US); Greg Marik, Germantown, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/040,565

(22) Filed: Jan. 21, 2005

(51) Int. Cl. A61F 2/44

(52) U.S. Cl. 623/17.11

(57) ABSTRACT

Described are artificial disc implants for insertion between first and second adjacent vertebrae in a patient. The implants have a first member and a second member in articulating relationship. At least one member of the implant includes an amorphous metal element. Also described are related methods of making and using artificial disc implants.

### BACKGROUND

The present invention relates generally to spinal implants, and in one particular aspect the invention relates to artificial spinal disc implants having one or more components made at least partially from amorphous metal.

As further background, diseases, injuries or malformations affecting spinal motion segments are commonly treated with intervertebral disc arthroplasty devices. The devices prevent the collapse of the intervertebral space between adjacent vertebrae while maintaining some degree of stability and range of pivotal and rotational motion therebetween. Intervertebral disc implants typically include two or more articular components that are attached to respective upper and lower vertebrae. These components are anchored to the upper and lower vertebrae, for example using bone screws, spikes, or teeth or other elements that penetrate the vertebral bodies to inhibit migration or expulsion of the device. The cooperating articular elements are typically configured to allow the elements, and in turn the adjacent vertebrae, to pivot and/or rotate relative to one another.

To date, intervertebral disc implants have included metallic components, sometimes in combination with plastic components. The metallic components often exhibit poor imaging characteristics under commonly used techniques such as x-ray, fluoroscopy, CT, and MRI imaging techniques. The materials used in the metallic components are typically highly radiopaque and tend to scatter radiation. This scattering can obscure peri-prosthetic tissue and make it more difficult to acertain the location and proper orientation of the implant. As well, this scattering can obscure details of the peri-prosthetic tissue that may be important to the physician, including for example soft or hard bone tissue position adjacent to the implant. This is a particular problem in the therapeutic treatment of the spine, where the proper alignment and position of the implant within an operative field containing and bounded by nervous tissue is needed.

In the case of articulating spinal implants with bearing surfaces, the wear characteristics of the components are of course also of prime importance. However, the provision of beneficial wear properties is commonly associated with relatively dense materials which have a higher tendency to scatter radiation and present poor imaging characteristics as discussed above.

In light of this background, there are needs for improved and/or alternative spinal disc devices with beneficial wear and imaging characteristics. The present invention is addressed to these needs.

### SUMMARY OF THE INVENTION

In one embodiment, the present invention provides an artificial disc implant for insertion between first and second adjacent vertebrae. The implant includes a first member having a surface configured to engage the first vertebra. The implant also includes a second member having a surface configured to engage the second vertebra. The first member is In articulating relationship with the second member, and includes a near net shape cast amorphous metal element presenting an articulation surface. The articulation surface can, for example, be either of convex or concave nature. As well, both the first and second members can include a near net shape cast amorphous metal element presenting an articulation surface.

In another embodiment, the invention provides an artificial disc implant for insertion between upper and lower vertebral bodies. The implant has a first member including a first main body for receipt between the upper and lower vertebral bodies. The first main body has an upper surface configured to engage an endplate of the upper vertebral body, and a lower surface presenting a convex articulation surface. The first member also includes an upwardly-extending portion connected to and extending upwardly from the main body. The upwardly-extending portion is configured to engage the upper vertebral body. The artificial disc implant also includes a second member having a second main body for receipt between the upper and lower vertebral bodies. The second main body has a lower surface configured to engage an endplate of the lower vertebral body, and an upper surface presented a concave articulation surface for movably contacting the convex articulation surface of the first member. The second member also includes a downwardly-extending member connected to and extending downwardly from the second main body. The downwardly-extending member is configured to engage the lower vertebral body. At least one of the convex articulation surface and concave articulation surface comprises an amorphous metal material, and in certain embodiments both of these articulation surfaces comprise an amorphous metal material. The upwardly- and downwardly-extending members can, as examples, be adapted to receive a connector such as a bone screw inserted into the corresponding vertebral bodies, and/or may be adapted to be received within the corresponding vertebral bodies.

The invention provides in another embodiment an artificial disc implant for insertion between adjacent vertebrae. The implant includes a first member made of amorphous metal and having a surface configured to engage a vertebra, and a second member made of amorphous metal having a surface configured to engage a vertebra. The first member is an articulating relationship with the second member.

In a further embodiment, the invention provides a method for making a component for an artificial disc implant. The method includes forming a near net shape amorphous metal element dimensioned for receipt between upper and lower vertebral bodies. The near net shape amorphous metal element includes a first surface configured to engage a vertebral endplate of the upper or lower vertebral body and a second surface presenting an articulation surface.

In additional embodiments, the present invention provides methods for treating the spine of a patient that involve implanting artificial disc implants of the invention.

In still further embodiments, the present invention provides kits for treating the spine of a patient that include a first member having a surface configured to engage a first vertebra of the spine. The first member desirably has a first engagement portion adapted to engage a connector extending into the first vertebra, and a near-net-shape cast amorphous metal element presenting a first articulation surface. The kit further includes a second member having a surface configured to engage the second vertebra of the spine. The second member desirably has a second engagement portion adapted to engage a connector extending into the second vertebra, and presents a second articulation surface for cooperation with the first articulation surface. The kit can further include at least one connector adapted to engage the first engagement portion, and at least one connector adapted to engage the second engagement portion.

Additional preferred embodiments as well as features and advantages of the invention will be apparent from the descriptions herein.

### DESCRIPTION OF THE FIGURES

FIG. 1 is a front elevational view of an embodiment of a ball component of an artificial disc implant of the invention.

FIG. 2 is a perspective view of an embodiment of a trough component of an artificial disc implant of the invention configured to cooperate with the component shown in FIG. 1.

FIG. 3 is a sectional view of an implant including the components shown in FIGS. 1 and 2.

FIG. 3a is a sectional view of an implant including alternative forms of the components shown in FIGS. 1 and 2.

FIG. 4 is a partial sectional view, showing a side view of an implant including the components shown in FIGS. 1 and 2 implanted between adjacent vertebrae shown in cross-section.

FIG. 5 is a perspective view of another artificial disc implant of the invention.

FIG. 6 is a sectional view of the artificial disc implant shown in FIG. 5.

FIG. 6a is a sectional view of an alternative form of the implant shown in FIG. 5.

FIG. 7 is a partial sectional view of a portion of a spinal column, illustrating implantation of the artificial disc implant of FIG. 5 between upper and lower vertebrae.

FIG. 8 is an anterior view of the portion of the spinal column shown in FIG. 7.

FIG. 9 is an exploded view of another artificial disc implant of the invention.

FIG. 10 is a sectional view of the implant of FIG. 9 implanted between upper and lower vertebrae.

FIG. 11 is a fragmentary side elevational view of the implanted artificial disc implant shown in FIG. 10.

### DETAILED DESCRIPTION

For the purposes of promoting an understanding of the principles of the invention, reference will now be made to certain embodiments thereof and specific language will be used to describe the same. It will nevertheless be understood that alterations and modifications in the described embodiments, and further applications of the principles of the invention as described herein are contemplated as would normally occur to one skilled in the art to which the invention relates.

As disclosed above, the present invention provides articulating artificial spinal disc implants, and methods for making and using such implants. Implants of the invention include articulating components, and at least one component of the implant is made at least in part of an amorphous metal material. Such artificial disc implants may be advantageously used in providing spinal therapy to patients. Amorphous metal materials have excellent strength and may be used to manufacture implants of low profile and bulk, and which exhibit beneficial imaging properties in compact and complicated operational windows in the spine in which visualization of implant placement and peri-prosthetic tissue is highly important. In addition, amorphous metal materials provide superior wear properties, low coefficients of friction, and artificial disc components of high integrity and reliability.

In this regard, a variety of amorphous metal materials are known and can be used in the present invention. As is known in the art, the term “amorphous metal” refers to a metal material that is non-crystalline in that it has substantially no long range order to the positions of the atoms of the material. The amorphous metal material can be, and typically is, an alloy. For example, amorphous metal materials that can be used in the invention include alloys containing titanium and/or zirconium.

One class of amorphous metal alloys that may be used includes alloys containing zirconium, titanium, beryllium, copper and nickel, and in particular (Zr,Ti)(Cu,Ni)Be alloy systems. Certain alloys that may be used are encompassed by the formula
(Zr₁₋ₓTiₓ)ₐ(Cu_{1-y}Ni_{y})_{b}Be_{c}
wherein:

x and y are atomic fractions ranging from 0 to 1;

a, b and c are atomic percentages, wherein
when x is in the range of from 0 to 0.15, a is in the range of from 30 to 75%, b is in the range of from 5 to 52%, and c is in the range of from 6 to 47%;
when x is in the range of from 0.15 to 0.4, a is in the range of from 30 to 75%, b is in the range of from 5 to 52%, and c is in the range of from 5 to 47%;
when x is in the range of from 0.4 to 0.6, a is in the range of from 35 to 75%, b is in the range of from 5 to 52%, and c is in the range of from 5 to 47%;
when x is in the range of from 0.6 to 0.8, a is in the range of from 38 to 75%, b is in the range of from 5 to 52%, and c is in the range of from 5 to 42%;
when x is in the range of from 0.8 to 1, a is in the range of from 38 to 75%, b is in the range of from 5 to 52%, and c is in the range of from 5 to 30%, under the constraint that 3c is up to (100-b) when b is in the range of from 10 to 43.

The (Zr₁₋ₓTiₓ) moiety may also comprise additional metal selected from the group consisting of from 0 to 25% hafnium, from 0 to 20% niobium, from 0 to 15% yttrium, from 0 to 10% chromium, from 0 to 20% vanadium, from 0 to 5% molybdenum, from 0 to 5% tantalum, from 0 to 5% tungsten, and from 0 to 5% lanthanum, lanthanides, actinium and actinides. The (Cu_{1-y} Ni_{y}) moiety may also comprise additional metal selected from the group consisting of from 0 to 25% iron, from 0 to 25% cobalt, from 0 to 15% manganese and from 0 to 5% of other Group 7 to 11 metals. The beryllium moiety may also comprise additional metal selected from the group consisting of up to 15% aluminum with the beryllium content being at least 6%, up to 5% silicon and up to 5% boron. Other elements in the composition should be less than two atomic percent.

Another class of bulk-soldifying amorphous alloy materials has a composition range, in atom percent, of from about 25 to about 85% total of zirconium and hafnium, from about 5 to about 35% aluminum, and from about 5 to about 70% total of nickel, copper, iron, cobalt, and manganese, plus incidental impurities, the total of the percentages being 100 atomic per cent. One specific metal alloy of this group has a composition, in atomic percent, of about 60% zirconium, about 15% aluminum, and about 25% nickel.

It is desirable that amorphous metal materials for use in the invention have the capacity to form substantially amorphous materials at cooling rates of less than 1000K/second. More desirably, cooling rates to avoid crystallization are in the range of from 1 to about 100K/second or lower. Additional information concerning these and other amorphous metal materials is found for example in U.S. Pat. Nos. 5,288,344, 5,032,196, 5,618,359, and 5,567,532. As well, amorphous metal alloys that may be used in the invention are commercially available from Liquidmetal Technologies.

In accordance with aspects of the invention, an articulating spinal disc implant will include at least one element made at least in part by an amorphous metal material. In this regard, it will be understood that one or more elements of the implant may be made completely from an amorphous metal material, or that selected portions of the component may be made from an amorphous metal material. Illustratively, a complete unitary element of the spinal implant can be cast to near net shape using the amorphous metal material, or may be otherwise formed as a monolithic element from the material including standard techniques such as machining, die stamping, and the like. Combinations of these techniques may likewise be used.

In certain embodiments, selected portions of the implant are made with an amorphous metal material. This may include, for example, one or more amorphous metal pieces that are connected to one or more other metal and/or plastic pieces, e.g. crystalline metal pieces made of materials such as titanium, steel, chromium-molybdenum alloys or other biocompatible materials commonly used in the fabrication of medical implants. In this regard, standard techniques can be used to connect amorphous and other (e.g. crystalline metal) elements, including for instance welding, brazing, bonding, mechanical connectors such as bolts or screws, and the like. Still further, an amorphous metal element can be mechanically locked to another element such as a crystalline metal element in a manner that involves the complete or partial formation of the amorphous metal element in, on, or in connection with the other element. Illustratively, a crystalline metal element can be used as a die in the molding or die casting of the amorphous metal element, wherein the crystalline metal element includes a depression for receiving amounts of the amorphous metal material as the amorphous metal material is formed. The depression can be configured so as to include recesses and overhangs so as to establish an interlocking connection between the formed amorphous metal element and the crystalline metal element. Illustratively, an amorphous metal element can be die-formed using a crystalline metal element as the die, wherein the crystalline metal element includes a re-entrant corner therein so as to mechanically lock the two elements together.

For example, a piece of bulk-solidifying amorphous alloy can be heated to a die-forming temperature, and then die-formed in connection with the crystalline metal element. The die-forming temperature may be from about 0.75 T_{g} to about 1.2 T_{g}, where T_{g} is measured in degrees C. For advantageous amorphous alloys, this temperature will be about 240° C. to about 385° C. Desirably, the die-forming temperature will be about 0.75 T_{g} to about 0.95 T_{g}. Upon being heated to the die-forming temperature, the piece of bulk-solidifying amorphous alloy is forced into the die cavity of the crystalline metal element so as to alter its shape to conform to the forming surface. In the case of a female die, the amorphous alloy piece is forced into the interior of the die cavity. When a re-entrant corner is present, the formed amorphous alloy element will be mechanically locked to the crystalline metal element used as the die such that it cannot be removed without destroying the formed amorphous metal element or the crystalline metal element. For additional information as to these techniques reference can be made for example to U.S. Pat. No. 5,896,642.

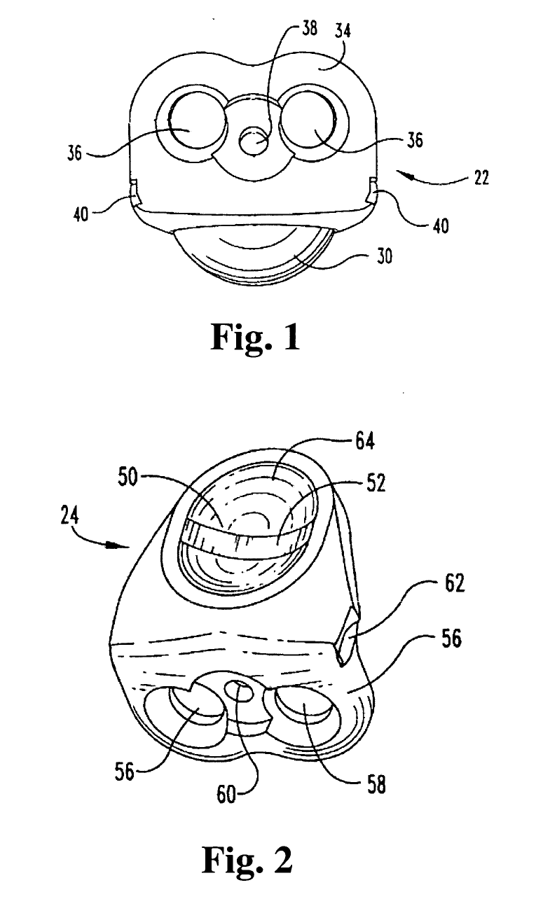

With reference now to FIGS. 1-4, one illustrative implant of the invention will now be described. Intervertebral disc prosthesis 20 includes a ball component 22 and a trough component 24 that are interengagable to form prosthesis 20. At least one of these elements 22 and 24 will be made at least partially from, and potentially completely from, an amorphous metal material.

In an intervertebral disc space 28 (FIG. 4) between two adjacent vertebrae 26, 27, ball component 22 is fixed to one of the adjacent vertebrae (e.g. vertebra 26 in FIG. 4), and trough component 24 is fixed to the other adjacent vertebra (e.g. vertebra 27 in FIG. 4) so that the components are interengaged within at least a portion of intervertebral space 28.

Ball component 22 includes a generally convex surface 30 and in one embodiment an opposite substantially flat vertebra engaging surface 32. In a particular embodiment generally convex surface 30 is substantially spherical in shape. In a specific embodiment, a wedge surface 33 is provided at one end of vertebra engaging surface 32, which allows easier insertion of ball component 22 into the disc space and impedes migration of ball component 22. A flange 34 is provided at one end of ball component 22 for attaching ball component 22 to a vertebra, and is preferably formed to have a low profile and bulk. In the embodiment in which ball component 22 includes wedge surface 33, flange 34 is at the opposite end of ball component 22 from wedge surface 33. Flange 34 includes a vertebra engaging surface 35.

In a particular embodiment of ball component 22, flange 34 includes one or more bone screw apertures 36, and in a specific embodiment, two bone screw apertures 36 are provided in a symmetric relationship through flange 34. In that specific embodiment, one or more bone screws 37 (FIG. 4) are threaded into the vertebra through one or more apertures 36 to fix ball component 22 to the vertebra. In the illustrated embodiment, an aperture 38 (FIG. 1) is also provided through flange 34 for a locking screw 39 (FIG. 4). After ball component 22 is attached to the vertebrae using bone screw(s) 37, locking screw 39 is threaded into locking screw aperture 38 in flange 34, covering the heads of bone screw(s) 37 and preventing loosening of bone screw(s) 37 with respect to ball component 22. Additionally, the illustrated embodiment of ball component 22 includes indentations 40 for gripping ball component 22 by an insertion tool. Preferably, indentations 40 are located at the base of flange 34, where flange 34 meets substantially flat vertebra engaging surface 32.

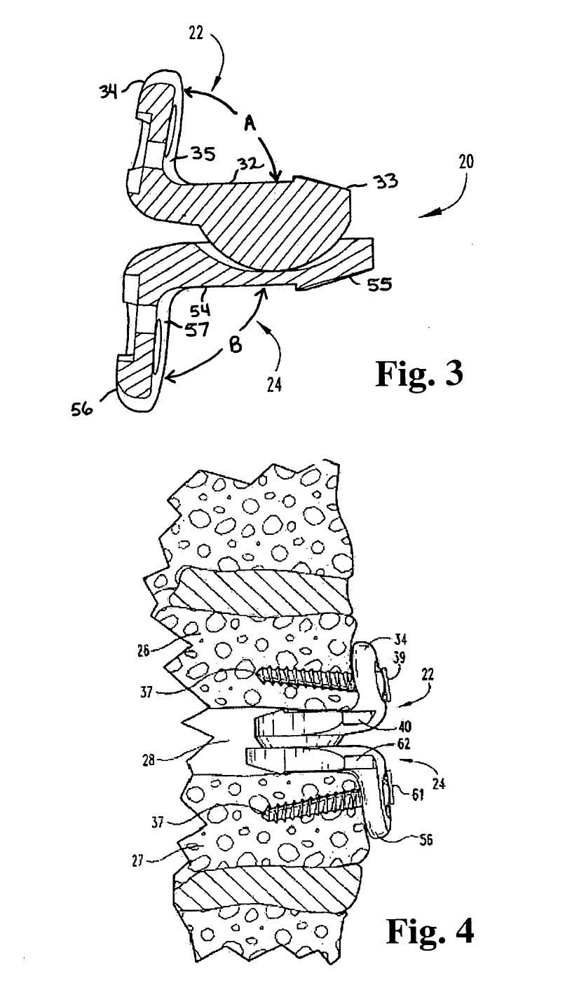

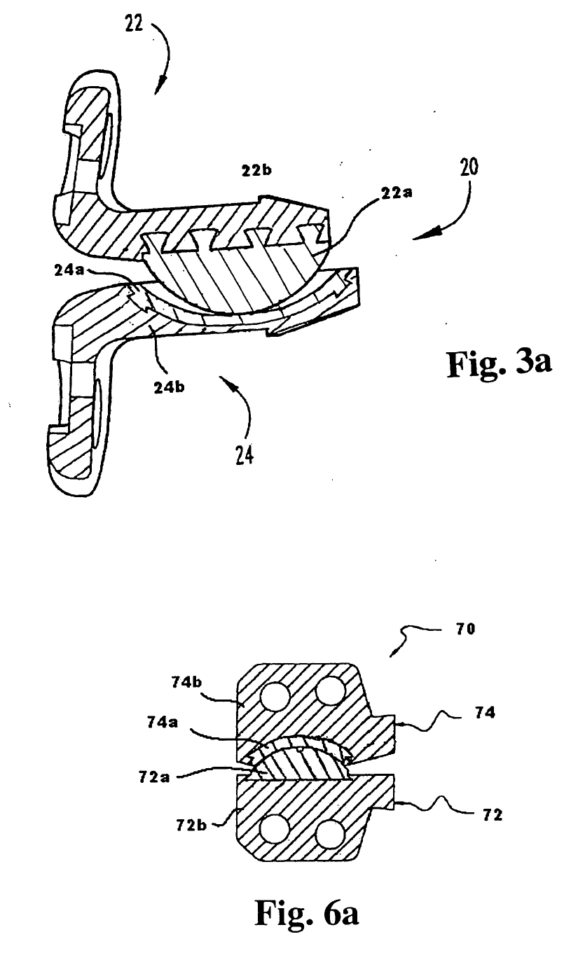

Flange 34 may be angled with respect to vertebra engaging surface 32 of ball component 22. In a particularly preferred embodiment, as illustrated in FIG. 3, the internal angle A between vertebra engaging surface 35 and vertebra engaging surface 32 is approximately 80 degrees. This angle has been found to provide a good fit with the anterior portion of the upper vertebra in a middle or lower cervical spinal motion segment, such as C4-C5. Ball component 22 can be constructed with a different angle between vertebra engaging surfaces 35 and 32 according to the vertebrae to be implanted and the needs of patients. For example, for upper cervical spinal motion segments such as C2-C3, the internal angle between vertebra engaging surface 35 and vertebra engaging surface 32 may be somewhat more acute, in the range of 70-80 degrees. Flange 34 may also be slightly curved from side to side, as illustrated in FIG. 3. Such curvature is particularly useful for anterior placement of prosthesis 20, as it approximates the lateral curvature of the anterior surface of the human vertebra.

Trough component 24, in the embodiment illustrated, is similar in many respects to ball component 22. Trough component 24 includes a generally concave surface 50, which generally concave surface 50 includes a substantially flat portion 52 (FIG. 2). Opposite generally concave surface 50 is a vertebra engaging surface 54, which, in the illustrated embodiment, includes a wedge surface 55 similar to wedge surface 33 of ball component 22. Trough component 24 also includes a flange 56 and a flange vertebrae engaging surface 57 which are similar to flange 34 and flange vertebra engaging surface 35 of ball component 22.

As with a particular embodiment of ball component 22, the illustrated embodiment of flange 56 of trough component 24 includes at least one aperture 58 and preferably two symmetric apertures 58, each of which can accommodate a bone screw. In that embodiment, flange 56 may also include a lock screw aperture 60 and lock screw 61 (FIG. 4), as described with respect to ball component 22. Additionally, trough component 24 in one embodiment includes indentations 62, which are similar in location and structure to indentations 40 of ball component 22. Flange 56 of trough component 24, in a preferred embodiment, is also angled with respect to vertebra engaging surface 54. In the illustrated embodiment, the internal angle B between flange vertebra engaging surface 57 and vertebra engaging surface 54 is approximately 95 degrees, which provides a good fit with the anterior portion of a lower vertebra of a middle or lower cervical spinal motion segment. As noted with respect to ball component 22, trough component 24 may be manufactured with a different angle between surfaces 57 and 54 according to the needs of the patient or other factors. For example, for upper cervical spinal motion segments, the angle between surfaces 57 and 54 may be between 90 and 100 degrees.

Generally concave surface 50, in the illustrated embodiment, includes a substantially flat (in that it is cylindrical rather than spherical) surface 52 that is approximately centrally located on generally concave surface 50. In a specific embodiment, generally concave surface 50 includes substantially spherical surfaces 64 on both sides of substantially flat surface 52. Substantially flat surface 52 may be of any desired geometrical configuration, though in a currently preferred embodiment, substantially flat surface 52 is in the shape of a rectangle, analogous to a slot in generally concave surface 50, and is approximately parallel to flange 56. In a particular preferred embodiment of ball component 22 and trough component 24, the radius of generally convex surface 30 and of the spherical portion of generally concave surface 50 is approximately the same.

In use, ball component 22 and trough component 24 are attached to adjacent vertebrae so that a portion of components 22, 24 are within the disc space and engage each other to provide a ball-and-socket arrangement which permits rotation and translation of the components, and thereby the vertebrae to which they are fixed with respect to each other. To accomplish that, when prosthesis 20 is implanted generally convex surface 30 of ball component 22 engages generally concave surface 50 of trough component 24. When engaged with generally concave surface 50, generally convex surface 30 can rotate in any direction. When generally convex surface 30 is in contact with substantially flat portion 52 of generally concave surface 50, generally convex surface 30 can move along substantially flat portion 52 in translation, as well as rotate with respect to generally concave surface 50. In a specific embodiment of prosthesis 20, ball component 22 is an “upper” component and is fixed to the vertebra immediately above or superior to the disc space (i.e. the vertebra closer to the head). In that embodiment, trough component 24 is attached to the lower or inferior vertebra (i.e. the vertebra closer to the coccyx). Thus, with ball component 22 attached to one vertebra and trough component 24 attached to an adjacent vertebra so that generally convex surface 30 and generally concave surface 50 are engaged, prosthesis 20 permits the adjacent vertebrae to rotate and translate with respect to each other, providing a normal range of intervertebral joint motion. For additional information concerning implants having the geometries discussed above, reference can be made for example to U.S. Pat. No. 6,540,785.

As noted above, either or both of components 22 and 24 may be made completely or partially from an amorphous metal material. With reference to FIG. 3a, shown is an embodiment in which the articulation surfaces of the ball 22 and trough 24 components are provided by amorphous metal pieces 22a and 24a that are mechanically connected to other (e.g. crystalline metal) elements 22b and 24b which may, for example, be fabricated from steel, titanium, chromium-molybdenum alloys, or other suitable materials. As shown in the embodiment illustrated in FIG. 3a, the mechanical connection of amorphous elements 22a and 24a can be made using a series of tapered-wall depressions within the non-amorphous metal elements 22b and 24b. In cross section these depressions appear in the nature of dovetail joint. Such a structure can be made for example in a die-forming procedure as discussed above in which an amorphous metal piece is die-formed into the crystalline metal pieces 22b and 24b which serve as the die in the procedure. The illustrated depressions include re-entrant corners or other like adaptations which serve to mechanically lock the amorphous and crystalline metal or other pieces together. It will be understood that other techniques for providing connected amorphous and crystalline metal pieces may be used including for example the use of connectors, bonding agents, coatings, welding, brazing, and the like.

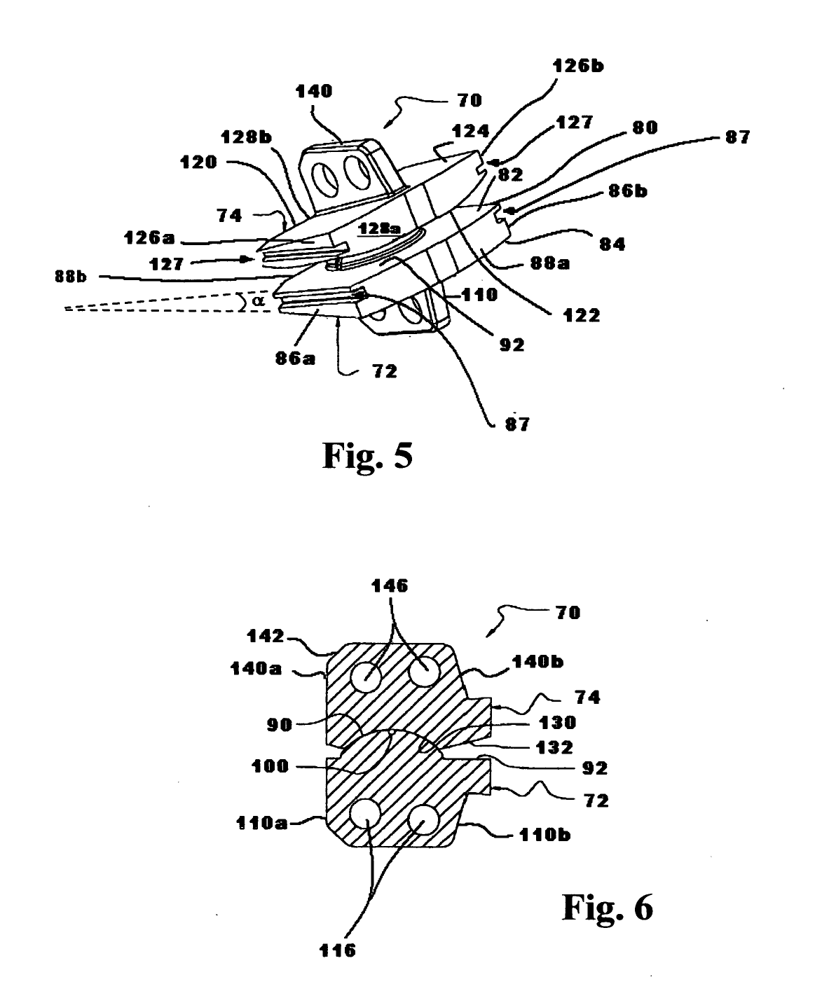

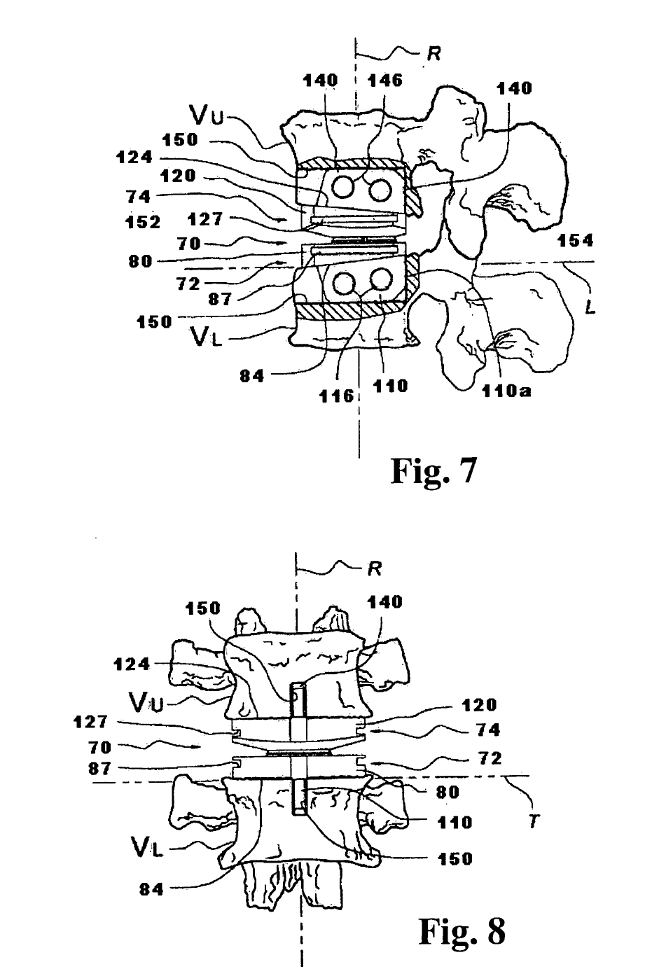

Referring now to FIGS. 5-8, shown therein is an artificial disc implant 70 according to another form of the present invention. The implant 70 extends generally along a longitudinal axis L (FIG. 7) and includes a first articular component 72 and a second articular component 74. The articular components 72, 74 cooperate to form the articulating implant 70 which is sized and configured for disposition within an intervertebral space between adjacent vertebral bodies.

The implant 70 provides relative pivotal and rotational movement between the adjacent vertebral bodies to maintain or restore motion substantially similar to the normal bio-mechanical motion provided by a natural intervertebral disc. More specifically, the articular components 72, 74 are permitted to pivot relative to one another about a number of axes, including lateral or side-to-side pivotal movement about longitudinal axis L and anterior-posterior pivotal movement about a transverse axis T (FIG. 8). It should be understood that in a preferred embodiment of the invention, the articular components 72, 74 are permitted to pivot relative to one another about any axes that lies in a plane that intersects longitudinal axis L and transverse axis T. Additionally, the articular components 72, 74 are preferably permitted to rotate relative to one another about a rotational axis R. Although the articulating joint 70 has been illustrated and described as providing a specific combination of articulating motion, it should be understood that other combinations of articulating movement are also possible and are contemplated as falling within the scope of the present invention. It should also be understood that other types of articulating movement are also contemplated, such as, for example, relative translational or linear motion.

The surfaces of the articular components 72, 74 that are positioned in direct contact with vertebral bone are adapted to promote bone ingrowth and attachment, and for these purposes can be coated with a bone-growth promoting substance, such as, for example, a calcium phosphate coating such as hydroxyapatite. Additionally, the surface of the articular components 72, 74 that are positioned in direct contact with vertebral bone are preferably roughened prior to being coated with the bone-growth promoting substance to further enhance bone on-growth. Such surface roughening may be accomplished by way of, for example, acid etching, knurling, application of a bead coating, or other methods of roughening that would occur to one of ordinary skill in the art.

Articular component 72 includes a support plate 80 having an articular surface 82 and an opposite bearing surface 84. Support plate 80 is preferably sized and shaped to substantially correspond to the size and shape of the vertebral endplate of an adjacent vertebra. The articular surface 82 and the bearing surface 84 are separated by a pair of laterally facing surfaces 86a, 86b and a pair of axially facing surfaces 88a, 88b. The laterally facing surfaces 86a, 86b each preferably define a channel 87 extending along at least a portion of the length of the support plate 80. The channels 87 are configured to engage a corresponding portion of a surgical instrument (not shown) to aid in the manipulation and insertion of the prosthetic joint 80 within an intervertebral space between adjacent vertebrae. The surgical instrument (not shown) is preferably configured to hold the articular components 72, 74 at a predetermined orientation and spatial relationship relative to one another during manipulation and insertion of the implant 70, and to release the articular components 72, 74 once properly positioned between the adjacent vertebrae.

In one embodiment of the invention, the articular surface 82 includes a projection 90 surrounded by a substantially planar surface 92. In one form, the projection 90 has a convex shape and can for example be configured as a spherical-shaped ball. In another embodiment of the invention, the spherical-shaped surface of the projection has a large enough radius of curvature such that the axis about which the articular components 72, 74 pivot relative to one another is located at or below the planar surface 92 (i.e., the center of curvature is located at or below planar surface 92). However, it should be understood that the pivot axis may alternatively be positioned above the planar surface 92. It should also be understood that other configurations of the projection 90 are also contemplated, such as, for example, cylindrical, elliptical or other arcuate configurations or possibly non-arcuate configurations. It should also be understood that the planar surface 92 may take on non-planar configurations, such as, for example, an angular or conical configuration extending about the projection 90.

In one embodiment of the invention, the convex articular surface of the projection 90 is interrupted by a surface depression or cavity 100 extending along the projection 90. In one form, the surface depression 100 is configured as a groove. However, as will be discussed in further detail below, it should be understood that other types of surface depressions are also contemplated. One purpose of the groove 100 is to facilitate the removal of matter disposed between abutting portions of the articular components 72, 74. More specifically, the groove 100 provides a means for clearing out matter such as, for example, particulate material, that is disposed between the abutting articular surfaces of components 72, 74.

In one embodiment, the groove 100 extends along the convex surface of the spherical-shaped ball 90 in such a manner as to divide the ball 90 into two substantially symmetrical portions, with each portion extending about approximately 180° of the overall circumference or periphery of the ball 90. However, it should be understood that the groove 100 may take on other configurations as well. For example, the groove 100 need not necessarily uniformly divide the ball 90 into symmetrical halves, but may alternatively be positioned at other locations along ball 90 and arranged at other angular orientations relative to ball 90. It should further be understood that the groove 100 need not necessarily extend entirely across the ball 90, but may alternatively extend across only a portion of the ball 90. For example, the groove 100 may extend across the ball 90 in such a manner that only a portion of the groove 90 extends beyond abutting portions of the articular components 72, 74 at some point during the articulating motion of joint 70. Additionally, it should be understood that the groove 100 need not necessarily have a linear configuration, but may alternatively take on angular configurations or non-linear configurations, such as, for example, the curvilinear configuration. It should also be understood that any number of grooves 100 may be defined along the periphery of the ball 90, such as two or more grooves 100 arranged in a uniform manner or alternatively in a random or semi-random pattern. In one specific embodiment of the invention, the groove 100 is approximately 0.75 mm deep and approximately 0.4 mm wide and has a radiused bottom surface. However, it should be understood that other sizes and configurations of the groove 100 are contemplated as falling within the scope of the present invention.

In one embodiment of the invention, the bearing surface 84 is substantially planar and is oriented at an angle a relative to the planar surface 92 to define an outward taper extending from axial surface 88b toward axial surface 88a. In certain embodiments, angle a falls within a range of 0 degrees to about 10 degrees. In a specific embodiment, angle α is about 3 degrees. In another specific embodiment, angle α is about 6 degrees. However, it should be understood that angle a may take on other values that correspond to the particular lordotic angle or morphology of the portion of the spinal column in which the implant 70 is used. It should further be understood that the bearing surface 84 may be configured to accommodate spinal abnormalities such as scoliosis. In such case, the bearing surface 84 may be angled relative to the planar surface 92 to define a taper extending between the lateral surfaces 86a, 86b. It should also be understood that the bearing surface 84 may take on alternative configurations, such as, for example, a curved or arcuate configuration that corresponds to the particular contour of the adjacent vertebral endplate against which surface 84 abuts. It should likewise be understood that bearing surface 84 may be roughened and/or may define a number of surface projections to aid in gripping the vertebral endplate and to inhibit migration of the prosthetic joint 70 relative to the adjacent vertebra.

A flange member or keel 110 extends from the bearing surface 84 and is configured for disposition within a preformed opening in the adjacent vertebral endplate. In one embodiment, the keel 110 extends perpendicularly from the bearing surface 84 and is approximately centrally located along the bearing surface 84. However, it should be understood that other positions and orientations of the keel 110 are also contemplated. It should also be understood that the articular component 72 may include two or more keels 110 extending from the bearing surface 84.

The keel 110 extends from a location adjacent the axially facing surface 88a toward the axially facing surface 88b along a substantial portion of the support plate 80. Preferably, the keel 110 extends along substantially the entire length of the support plate 80. The keel 110 is preferably wedge-shaped, defining an outward taper as the keel 110 extends from a leading or insertion end 110a towards a trailing end 110b. In one specific embodiment, the outward taper is about 4 degrees. However, other taper angles are also contemplated. It should also be understood that the keel 110 need not necessarily be tapered along it length. As will become apparent, the outward taper aids in the insertion of the keel 110 within preformed openings in the adjacent vertebrae. Additionally, the insertion end 110a of keel 110 includes a beveled surface 112 to further aid in the implantation of the prosthetic joint 70.

In another embodiment of the invention, the keel 110 may alternatively extend between the laterally facing surface 86a, 86b along a substantial portion of the support plate 80. Such an embodiment would accommodate insertion of the implant 70 using a lateral approach as opposed to an anterior approach. In a further embodiment of the invention, the keel 110 may be tapered along its height, either tapering inwardly from bearing surface 84 to define a wedge shape or tapering outwardly from bearing surface 84 to define a dove-tail shape. In still another embodiment, the keel 110 may be configured as a winged keel, including a transverse portion extending across the main body portion of keel 110.

The keel 110 also includes a pair of openings 116 extending therethrough to facilitate bone through-growth to enhance fixation to the adjacent vertebra. However, it should be understood that any number of openings 116 may be defined through keel 110, including a single opening or three or more openings. It should also be understood that the openings 116 need not necessarily extend entirely through the keel 110, but may alternatively extend partially therethrough. It should further be understood that the keel 110 need not necessarily define any openings 116 extending either partially or entirely therethrough. Additionally, although the openings 116 are illustrated as having a circular configuration, it should be understood that other sizes and configures of openings 116 are also contemplated. As discussed above, the surfaces of the articular component 72 that are in direct contact with vertebral bone can be coated with a bone-growth promoting substance. Specifically, the bearing surface 84 and the surfaces of the keel 110 can be coated with hydroxyapatite to promote bony engagement with the adjacent vertebrae. As also discussed above, the bearing surface 84 and the surfaces of keel 110 are preferably roughened prior to application of the hydroxyapatite coating.

Articular component 74 includes a support plate 120 having an articular surface 122 and an opposite bearing surface 124. Support plate 120 is preferably sized and shaped to substantially correspond to the size and shape of the vertebral endplate of an adjacent vertebra. The articular surface 122 and the bearing surface 124 are separated by a pair of laterally facing surfaces 126a, 126b and a pair of axially facing surfaces 128a, 128b. The laterally facing surfaces 126a, 126b each preferably define a channel 127 extending along at least a portion of the length of the support plate 120. Similar to channels 87 of articular element 72, channels 127 are configured to engage a corresponding portion of a surgical instrument (not shown) to aid in the manipulation and insertion of the prosthetic joint 70.

In an advantageous embodiment of the invention, the articular surface 122 includes a recess 130 surrounded by a substantially conical surface 132. In one embodiment of the invention, the recess 130 has a concave shape, and is preferably configured as a spherical-shaped socket. However, it should be understood that other configurations of the recess 130 are also contemplated, such as, for example, cylindrical, elliptical or other arcuate configurations or possibly non-arcuate configurations. Conical surface 132 is tapered at an angle θ relative to a plane oriented parallel with the planar surface 82 of articular component 72 in such a manner as to define a uniform taper extending entirely about the concave recess 130. In this manner, relative pivotal motion between the articular components 72, 74 is limited to approximately ±angle θ. In one embodiment, the angle θ falls within a range of about 10 degrees to about 20 degrees, thereby limiting the overall relative pivotal motion between the articular components 72, 74 within a range of just over 20 degrees to just over 40 degrees. In a specific embodiment, angle θ is about 16 degrees, thereby limiting the overall pivotal motion between the articular components 72, 74 to just over 32 degrees. As will become apparent, angle θ may take on other values that correspond to the desired amount of relative pivotal movement between the articular components 72, 74. It should also be understood that the conical surface 132 may take on other configurations, such as, for example, an angular configuration extending about the concave recess 130. It should also be understood that the surface 132 could alternatively be configured as a planar surface oriented parallel with the bearing surface 124, and that the surface 82 of articular component 72 could alternatively be configured as a conical or angled surface tapered at an angle θ, or that both of the surfaces 82, 132 could alternatively be configured as conical or angled surfaces tapered at a predetermined angle θ. In an embodiment where both of the surfaces 82, 132 are tapered at a predetermined angle θ, the angle θ is preferably about 8 degrees, thereby limiting the overall pivotal motion between the articular components 72, 74 to just over 32 degrees.

Although the concave recess 130 is illustrated as having a generally smooth, uninterrupted articular surface, it should be understood that a surface depression or cavity may be defined along a portion of the recess 130 to provide a means for clearing out matter, such as particulate debris, that is disposed between the abutting articular surfaces of components 72, 74. In such case, the convex articular surface of the ball 90 may alternatively define a generally smooth, uninterrupted articular surface. In another embodiment of the invention, each of the convex projection 90 and the concave recess 130 may define a surface depression to facilitate removal of particulate matter disposed between the abutting articular surfaces.

In one embodiment of the invention, the bearing surface 124 is substantially planar and is oriented at an angle α, similar to that of bearing surface 84 of articular component 72, to define an outward taper extending from axial surface 128a toward axial surface 128b. However, it should be understood that bearing surface 124 may take on alternative configurations, such as, for example, a curved or arcuate configuration that corresponds to the particular contour of the adjacent vertebral endplate against which surface 124 abuts. It should further be understood that the bearing surface 124 may be configured to accommodate spinal abnormalities such as scoliosis. In such case, the bearing surface 124 may be angled to define a taper extending between the lateral surfaces 126a, 126b. It should additionally be understood that the bearing surface 124 may be roughened and/or may define a number of surface projections to aid in gripping the vertebral endplate and to inhibit migration of the prosthetic joint 70 relative to the adjacent vertebra.

A flange member or keel 140, configured similar to the keel 110 of articular component 72, extends from the bearing surface 124. In one embodiment, the keel 140 extends perpendicularly from the bearing surface 124 and is approximately centrally located along bearing surface 124. However, it should be understood that other positions and orientations of the keel 140 are also contemplated. It should also be understood that the articular component 74 may include two or more keels 140 extending from the bearing surface 124.

The keel 140 extends from a location adjacent axially facing surface 128a toward axially facing surface 128b, preferably along a substantial portion of the support plate 120. The keel 140 is preferably wedge-shaped, defining an outward taper as the keel 140 extends from a leading or insertion end 140a to trailing end 140b. Additionally, the insertion end 140a of keel 140 includes a beveled surface 142 to further aid in the implantation of the prosthetic joint 70. In another embodiment of the invention, the keel 140 may alternatively extend between the laterally facing surface 126a, 126b along a substantial portion of the support plate 120 to accommodate for insertion of the prosthetic joint 70 between adjacent vertebral bodies using a lateral approach. In a further embodiment of the invention, the keel 140 may be tapered along its height, either tapering inwardly from the bearing surface 124 to define a wedge shape or tapering outwardly from bearing surface 124 to define a dove-tail shape. In still another embodiment, the keel 140 may be configured as a winged keel, including a transverse portion extending across the main body portion of keel 120.

Keel 140 includes a pair of openings 146 extending therethrough to facilitate bone through-growth to enhance fixation to the adjacent vertebra. However, it should be understood that any number of openings 146 may be defined through the keel 140, including a single opening or three or more openings. It should also be understood that the openings 146 need not necessarily extend entirely through keel 140, but may alternatively extend partially therethrough. It should further be understood that the keel 140 need not necessarily define any openings 146 extending either partially or entirely therethrough. As discussed above, the surfaces of the articular component 34 that are in direct contact with vertebral bone are preferably coated with a bone-growth promoting substance, such as, for example, a hydroxyapatite coating. As also discussed above, the surfaces of the articular component 74 that are in direct contact with vertebral bone are preferably roughened prior to application of the bone-growth promoting substance.

The projection or ball 90 of articular component 72 is at least partially disposed within the recess or socket 130 of articular component 74. The convex and concave articular surfaces of ball 90 and socket 130 abut one another in such a manner as to provide relative articulating motion between the articular components 72, 74. Specifically, the articular components 72, 74 are allowed to pivot and rotate relative to one another to maintain or restore motion substantially similar to the normal biomechanical motion provided by a natural intervertebral disc. The relative pivotal motion between the articular components 72, 74 is limited by the abutment of the conical surface 132 of component 74 against the planar surface 92 of component 72. During the articulating motion, the groove 100 formed along the ball 90 provides a passage for removing any matter, such as particulate debris, that may become lodged between the abutting articular surfaces of the components 72, 74. The groove 100 channels any such debris clear from the interfacing articular surfaces of the prosthetic joint 70 to prevent or at least reduce wear which otherwise might occur if foreign particles and/or built-up wear debris were to remain between the abutting portions of the articular surfaces.

Referring to FIGS. 7 and 8, following preparation of the intervertebral space, the articular components 72, 74 are inserted between the upper and lower vertebrae V_{U}, V_{L}. First, the articular components 72, 74 are placed in a predetermined relationship with respect to one another, preferably by an insertion instrument (not shown) or an equivalent tool that is adapted to engage the channels 87, 127 formed along a length of the support plates 80, 120. The insertion instrument (not shown) holds the articular components 72, 74 in a predetermined spatial relationship and at a predetermined orientation with respect to one another. The prosthetic joint 70 is inserted between the upper and lower vertebrae V_{U}, V_{L} in a direction generally along the longitudinal axis L, with the keels 110, 140 of components 72, 74 being axially displaced along the slots 150. Notably, since the keels 110, 140 are axially displaced through the preformed slots 150, distraction of the upper and lower vertebrae V_{U}, V_{L} to accommodate insertion of the prosthetic joint 70 is minimized, if not eliminated entirely.

As discussed above, the keels 110, 140 are tapered or wedge-shaped to facilitate insertion within the slots 150. The taper angle defined by each of the support plates 80, 120 also facilitates insertion of the prosthetic joint 70 within the intervertebral space. Since the width of the slots 150 is equal to or somewhat less than the corresponding width of the keels 110,140, the keels are effectively wedged within the slots 150. The depth of the slots 150 formed in the upper and lower vertebrae V_{U}, V_{L} correspondingly controls the positioning of the implant 70 within the intervertebral space. Specifically, proper positioning of the implant 70 is accomplished when the insertion ends 110a, 140a of the keels 110, 140 bottom out against the end surfaces of slots 150. Controlling the insertion depth of the implant 70 results in more precise positioning to avoid over-insertion or under-insertion of prosthetic joint 70. As discussed above, the angular positioning of the articular components 72, 74 relative to one another is dictated by the geometry of the upper and lower vertebrae V_{U}, V_{L} and the particular location within the spinal column. As should be apparent, the distance between the support plates 80, 120 should be approximately equal to the height of the removed disc, and the angular disposition of the support plates 80, 120 is dictated by the particular curvature or lordosis of the spinal column.

Once the implant 70 is inserted within the intervertebral space, the articular components 72, 74 are initially secured to the upper and lower vertebrae V_{U}, V_{L} via the disposition of the keels 110, 140 within the slots 150 formed in the vertebrae V_{U}, V_{L} and by the compression forces exerted upon the bearing surfaces 84, 124 of the articular components 72, 74 by the adjacent vertebral endplates. The keels 110, 140 thus serve to resist migration or displacement of the prosthetic joint 70 relative to the adjacent vertebrae V_{U}, V_{L} Subsequent to the implantation of prosthetic joint 70, the articular components 72, 74 are further secured to the upper and lower vertebrae V_{U}, V_{L} via bone growth through the openings 116, 146 in keels 110, 140 and/or by bone on-growth onto the surfaces of the articular components 72, 74 that are in direct contact with vertebral bone. The bone through-growth and bone on-growth provide further resistance to the migration or displacement of the implant 70 and prevent expulsion of the implant 70 from the intervertebral space. It should be understood that other means of engaging the implant 70 to the upper and lower vertebrae V_{U}, V_{L} are also contemplated, such as, for example, by bone screws, staples, an adhesive, or by other methods of engagement as would occur to one of ordinary skill in the art.

In use, the articular components 72, 74 cooperate with one another to provide a ball-and-socket type joint that permits relative pivotal and rotational movement therebetween, which correspondingly permits relative pivotal and rotational movement between the upper and lower vertebrae V_{U}, V_{L} As a result, substantially normal biomechanical motion is restored to the portion of the spinal column being treated. Although the devices and methods of the present invention are particularly applicable to the lumbar region of the spine, it should nevertheless be understood that the present invention is also applicable to other portions of the spine, including the cervical or thoracic regions of the spine. For additional information as to artificial disc implants of the type discussed herein in conjunction with FIGS. 5-8, reference can be made for example to U.S. Pat. No. 6,740,118.

As noted above, all or one or more portions of component 72 and/or component 74 can be made of an amorphous metal material. With reference to FIG. 6a shown is an alternative arrangement similar to that depicted to that in FIG. 3a, in which the articulating surfaces of the artificial disc implant 70 are presented by an amorphous metal element connected to a crystalline metal element. Specifically, component 72 is fabricated as a combination of amorphous metal element 72a and crystalline metal element 72b, and component 74 is made as a combination of amorphous metal element 74a and crystalline metal element 74b. In each case, the amorphous metal element 72a or 74a is mechanically interlocked to its corresponding crystalline metal element 72b or 74b. This interlocking can for example be achieved using a die-forming technique using the crystalline metal elements as die pieces as generally discussed above. As well, it will be understood that additional embodiments of the invention are provided wherein only one of the components 72 or 74 includes an amorphous metal insert in combination with a crystalline metal element, and the other corresponding element 72 or 74 is made entirely of crystalline or amorphous metal material or another suitable material.

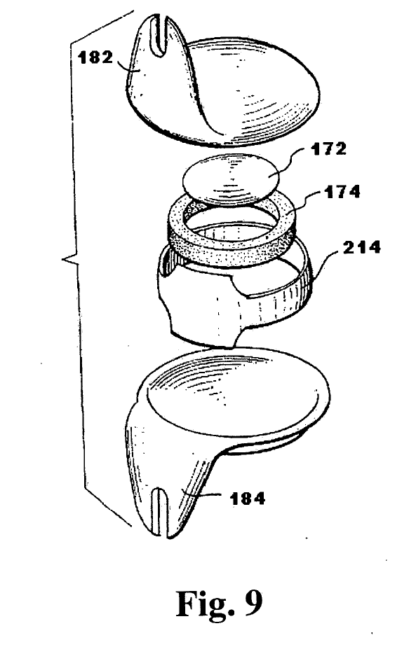

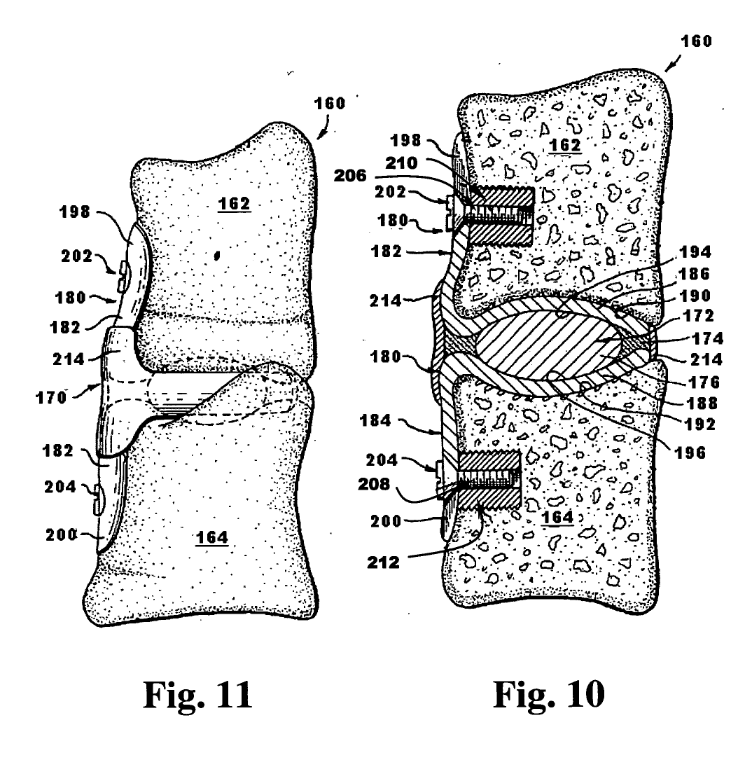

Referring now to FIGS. 9-11, a further artificial disc implant of the invention will be described. The illustrated spine 160 (FIGS. 10 and 11) has been subjected to a discectomy surgical process. To discourage degeneration of or damage to the natural vertebral bodies 162 and 164 and their respective facet joints, in accordance with the invention, an implant 170 is affixed between the adjacent natural vertebral bodies 162 and 164. Here this implant 170 comprises a disc body 172 having an annular gasket exterior portion 174 and a nuclear central portion 176. The annular gasket 174 can be formed from a suitable polymeric material. Likewise, the nuclear central portion 176 can be formed from a suitable polymeric material. Illustratively, nuclear central portion 176 can be made from ultra high molecular weight (UHMW) polyethylene.

Concaval-convex means 180 surround the resilient body 172 to retain the resilient body 172 between the adjacent natural vertebral bodies 162, 164 in a patient's spine. To this end, the concaval-convex means 180 comprise two generally L-shaped supports 182 and 184. The supports 182, 184 each have confronting first concaval-convex legs 186, 188, each leg being of relatively constant cross-sectional thickness. Each leg 186, 188 has an outer convex surface 190, 192 for engaging the adjacent bone of the natural vertebral bodies 162, 164. Corresponding inner concave surfaces 194, 196 in confronting array retain the resilient body 172 in its illustrated compressive force shock-absorbing position. These supports 182 and 184 can undergo principle movement away from one another, but only limited secondary translational, rotational and distractional motion will occur. Each support 182, 184 has a second wing or leg 198, 200 extending generally perpendicularly to the first legs 186, 188 respectively, and adapted for affixation to the adjacent bone structure. The affixation can be accomplished by screw devices 202, 204. Each device 202, 204 may comprise a screw 206, 208; and a screw anchor 210, 212 adapted to threadably receive the screw extends radially into and seats within the bone structure 162, 164.

To discourage and prohibit migration of fluids between the implant 170 and adjacent parts of the anatomy, a seal member 214 is attached to the supports 182, 184 so as to surround the body 172 comprised of the gasket 174 and nucleus 176. This seal member 214 may comprise a flexible sheet material having a multiplicity of pores. A flexible, strong polymer sheet material from which this seal is formed can be a Kevlar-like material, or it can be Goretex-like material, or other appropriate biocompatible material, such as polyether, polyurethane, or polycarbonate urethane membranes, can be used. Kevlar material is offered by the E. I. DuPont de Nemours Company of Wilmington, Del. and Goretex material is offered by the W. T. Gore Company of Flagstaff and Phoenix, Ariz. Known sealing material can be applied to the flexible sheet material so as to render the flexible sheet material substantially impervious to the passage of any fluid. A watertight seal is perfected when the seal 214 is glued or otherwise affixed to the legs 186, 188 and mediate portions of the legs 198, 200. For additional information as to implants having geometries as discussed herein in connection with FIGS. 9-11, reference can be made for example to U.S. Pat. No. 5,674,296.

In accordance with certain embodiments of the invention, all or a portion of one or both of components 182 and 184 can be made from an amorphous metal material. Still further, in addition to or as an alternative to one or both of components 182 and 184 being made from amorphous metal material, central core 172 may also be made completely or in part from an amorphous metal material. Portions of implant 170 that are not made with an amorphous metal material can be made, for example, of suitable crystalline metal or plastic.

Artificial disc implants of the invention, incorporating an element made from an amorphous metal material, are advantageously used in providing spinal therapy to patients. Amorphous metal materials provide superior wear properties and low coefficients of friction. Importantly also, amorphous metal materials exhibit very high strength and thus may be used to provide reliable spinal disc components with high integrity, using a minimum of metal material; this, in combination with their amorphous character may be used to provide beneficial imaging properties when employing conventional imaging techniques, during which scattering of the energy used in the creation of the image can be reduced or minimized. This will improve the ability to visualize the positioning of the implant and the presence and character of peri-implant tissue in the operative window.

While the invention has been illustrated and described in detail in the foregoing description, the same is to be considered as illustrative and not restrictive in character, it being understood that only certain embodiments have been shown and described and that all changes and modifications that fall within the spirit of the invention are desired to be protected. In addition, all publications cited herein are hereby incorporated by reference in their entirety as if each had been individually incorporated by reference and fully set forth.

What is claimed is:

1. An artificial disc implant for insertion between a first and second adjacent vertebrae, comprising:
a first member having an engagement surface configured to engage the first vertebra;
a second member having an engagement surface configured to engage the second vertebra;
said first member in articulating relationship with said second member; and
said first member comprising a near-net-shape cast amorphous metal element presenting a first articulation surface.

2. The artificial disc implant of claim 1, wherein:
said first articulation surface is convex.

3. The artificial disc implant of claim 2, wherein:
said second member presents a second articulation surface for cooperation with said first articulation surface, wherein said second articulation surface is concave.

4. The artificial disc implant of claim 1, wherein:
said first articulation surface is concave.

5. The artificial disc implant of claim 4, wherein:
said second member presents a second articulation surface for cooperation with said first articulation surface, wherein said second articulation surface is convex.

6. The artificial disc implant of claim 1, wherein:
said second member presents a second articulation surface; and
said implant also comprises a third member received between said first and second members and presenting articulation surfaces for cooperation with said first and second articulation surfaces.

7. The artificial disc implant of claim 6, wherein:
said first articulation surface is concave;
said second articulation surface is concave; and
said third member comprises a polymeric material and presents convex articulation surfaces for cooperation with said first and second articulation surfaces.

8. The artificial disc implant of claim 6, wherein:
said articulation surface of said third member is concave, and said articulation surface of said first member is convex.

9. The artificial disc implant of claim 1, wherein:
said first member includes a surface configured to engage a vertebral endplate of a first vertebral body, and a connector for connection to said first vertebral body; and
said second member includes a surface configured to engage a vertebral endplate of a second vertebral body, and a connector for connection to said second vertebral body.

10. The artificial disc implant of claim 9, wherein:
said first articulation surface is convex;
said second member presents a second articulation surface for cooperation with said first articulation surface, wherein said second articulation surface is concave.

11. The artificial disc implant of claim 10, wherein said first articulation surface is comprised of amorphous metal.

12. The artificial disc implant of claim 10, wherein said second articulation surface is comprised of amorphous metal.

13. The artificial disc implant of claim 1, wherein:
said engagement surfaces are adapted to promote bone attachment.

14. The artificial disc implant of claim 1, wherein:
said first member has a first portion and a second portion, said first portion made of amorphous metal and said second portion made of crystalline metal.

15. The artificial disc implant of claim 14, wherein:
said first portion provides said articulation surface comprised of amorphous metal.

16. The artificial disc implant of claim 15, wherein:
said first portion is a coating of amorphous metal on said second portion.

17. The artificial disc implant of claim 16, wherein:
said first portion is an element connected to said second portion.

18. The artificial disc implant of claim 17, wherein:
said first portion is mechanically interlocked with said second portion.

19. The artificial disc implant of claim 1, which is a cervical disc implant.

20. The artificial disc implant of claim 1, which is a lumbar disc implant.

21. The artificial disc implant of claim 1, wherein:
said first member is a monolithic near-net-shape cast amorphous metal element having an articulation surface.

22. The artificial disc implant of claim 21, wherein:
said second member is a monolithic near-net-shape cast amorphous metal element.

23. The artificial disc implant of claim 1, wherein:
said amorphous metal is a titanium-containing alloy.

24. The artificial disc implant of claim 1, wherein:
said amorphous metal is a zirconium-containing alloy.

25. The artificial disc implant of claim 1, wherein:
said amorphous metal is a metal alloy containing zirconium, titanium, beryllium, copper and nickel.

26. An artificial disc implant for insertion between upper and lower vertebral bodies, comprising:
a first member having a first main body for receipt between the upper and lower vertebral bodies, the first main body having an upper surface configured to engage an endplate of the upper vertebral body, said first main body having a lower surface presenting a convex articulation surface, said first member further comprising an upwardly-extending portion connected to and extending upwardly from said main body, said upwardly-extending portion configured for attachment to the upper vertebral body;
a second member having a second main body for receipt between the upper and lower vertebral bodies, the second main body having a lower surface configured to engage an endplate of the lower vertebral body, said second main body having an upper surface presenting a concave articulation surface for movably contacting said convex articulation surface, said second member further comprising a downwardly-extending member connected to and extending downwardly from said second main body, said downwardly-extending member of said second member configured for attachment to the lower vertebral body; and
at least one of said convex articulation surface and said concave articulation surface comprised of an amorphous metal material.

27. The artificial disc implant of claim 26, wherein:
at least one of said first and second members is a monolithic near-net-shape cast amorphous metal element.

28. The artificial disc implant of claim 26, wherein:
said first and second members are both monolithic near-net-shape cast amorphous metal elements.

29. The artificial disc implant of claim 26, wherein:
said amorphous metal is a titanium-containing alloy.

30. The artificial disc implant of claim 26, wherein:
said amorphous metal is a zirconium-containing alloy.

31. The artificial disc implant of claim 26, wherein:
said amorphous metal is a metal alloy containing zirconium, titanium, beryllium, copper and nickel.

32. An artificial disc implant for insertion between adjacent vertebrae, comprising:
a first member made of amorphous metal and having a surface configured to engage a vertebra;
a second member made of amorphous metal having a surface configured to engage a vertebra; and
said first member in articulating relationship with said second member.

33. A method for making a component for an artificial disc implant, comprising:
forming a near-net-shape amorphous metal element dimensioned for receipt between upper and lower vertebral bodies, said near-net-shape amorphous metal element including a first surface configured to engage a vertebral endplate of the upper or lower vertebral body and a second surface presenting an articulation surface.

34. The method of claim 33, wherein said articulation surface is concave.

35. The method of claim 33, wherein said articulation surface is convex.

36. The method of claim 33, wherein:
said near net-shape amorphous metal element has a main body portion, said main body portion including an upper surface configured to contact a the upper vertebral body and a lower surface presenting said articulation surface, said near-net-shape element also including an upwardly-extending portion connected to and extending upwardly from said main body portion, said upwardly-extending portion configured for attachment to the upper vertebral body.

37. The method of claim 36, wherein:
said upwardly extending portion is configured to extend upwardly along an anterior surface of the upper vertebral body and to engage a connector extending into the upper vertebral body.

38. The method of claim 33, wherein:
said near net-shape amorphous metal element has a main body portion, said main body portion including a lower surface configured to contact the lower vertebral body and an upper surface presenting said articulation surface, said near-net-shape element also including an downwardly-extending portion connected to and extending downwardly from said main body portion, said downwardly-extending portion configured for attachment to extend downwardly along an anterior surface of said lower vertebral body and to engage a connector extending into the lower vertebral body.

39. The method of claim 38, wherein:
said downwardly-extending portion is configured to extend downwardly along an anterior surface of said lower vertebral body and to engage a connector extending into the lower vertebral body.

40. The method of claim 33, wherein:
said amorphous metal is a titanium-containing alloy.

41. The method of claim 33, wherein:
said amorphous metal is a zirconium-containing alloy.

42. The method of claim 33, wherein:
said amorphous metal is a metal alloy containing zirconium, titanium, beryllium, copper and nickel.

43. A method for treating the spine of a patient, comprising:
implanting an artificial disc implant according to claim 1 between first and second adjacent vertebrae of the spine of a patient.

44. A method for treating the spine of a patient, comprising:
implanting an artificial disc implant according to claim 26 between upper and lower vertebral bodies of the spine of the patient.

45. A kit for treating the spine of a patient, comprising:
a first member having a surface configured to engage a first vertebra of the spine and including a first engagement portion adapted to engage a connector extending into the first vertebra, said first member including a near-net-shape cast amorphous metal element presenting a first articulation surface;
a second member having a surface configured to engage a second vertebra of the spine and having a second engagement portion adapted to engage a connector extending into the second vertebra, said second member presenting a second articulation surface for cooperation with said first articulation surface; and
at least one connector adapted to engage said first engagement portion; and
at least one connector adapted to engage said second engagement portion.
